(19) United States
(12) Patent Application Publication
(10) Pub. No.: US 2026/0242313 A1
(43) Pub. Date: Aug. 20, 2026

(54) METHANOL SYNTHESIS WITH HYDROGEN RECOVERY UNIT

(71) Applicant: TOPSOE A/S, Kgs. Lyngby (DK)

(72) Inventors: Søren Grønborg ESKESEN, Kgs. Lyngby (DK); Per Aggerholm SØRENSEN, Kgs. Lyngby (DK)

(73) Assignee: TOPSOE A/S, Kgs. Lyngby (DK)

(21) Appl. No.: 19/160,921

(22) PCT Filed: Apr. 2, 2024
(86) PCT No.: PCT/EP2024/058861

§ 371 Date: Aug. 29, 2025

(30) Foreign Application Priority Data
Apr. 3, 2023 (EP) 23166302.2

(51) Int. Cl. C07C 29/151; C07C 29/152; C07C 29/80; C07C 31/04; C25B 1/04

(52) U.S. Cl. CPC C07C 29/1518; C07C 29/152; C07C 29/80; C07C 31/04; C25B 1/04

(57) ABSTRACT

A methanol plant and a process for the production of methanol is provided. A hydrogen recovery section receives off-gas stream from the methanol synthesis section and outputs a hydrogen-rich stream, which is recycled upstream the methanol synthesis section.

### TECHNICAL FIELD

The present invention relates to a methanol plant and a process for the production of methanol. A hydrogen recovery section receives off-gas stream from the methanol synthesis section and outputs a hydrogen-rich stream, which is recycled upstream the methanol synthesis section.

### BACKGROUND

Producing methanol from mixtures of CO₂ and H₂ is facing increased focus due to climate issues, and an effort to bring CO₂ by-products from other processes into use. A straightforward route for methanol production is to mix CO₂ with H₂ directly and react this in a methanol loop (or similar methanol synthesis process technology) according to the reaction:

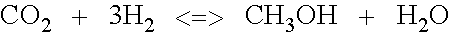

Herein referred to as CO₂-to-MeOH.

In conventional processes, the hydrogen required for the methanol synthesis may be obtained through electrolysis of water to hydrogen. Typically, such an electrolysis step requires large amounts of electrical energy, and may consume the majority of the power in conventional systems (typically above 90%).

Therefore, it would be very beneficial to limit the amount of hydrogen amount required from the electrolysis, and thereby, reducing the amount of electrical power required for this step.

It is an object of embodiments of the invention to provide a process and a plant for methanol synthesis from CO₂ and H₂, in which electrical power consumption can be reduced.

Prior art in this field includes WO2021156179 and WO2022248434.

US2021/363079 discloses a process for producing methanol comprising providing a CO₂ feed and a H₂ feed from an electrolyser, mixing the feeds and feeding the mixture to a methanol synthesis section to produce raw methanol, and purifying the raw methanol in a distillation unit.

### SUMMARY

It has been found by the present inventor(s) that the amount of power required in the electrolysis step can be reduced by recycling of certain components.

So, in a first aspect, a methanol plant is provided, said methanol plant comprising:
a first feed comprising CO₂ to said plant,
a second feed comprising water to said plant,
an electrolysis section and a source of electrical power to said electrolysis section, said electrolysis section being arranged to electrolyse the second feed into a first hydrogen-rich stream,
a compression section arranged to receive said first hydrogen-rich stream and said first feed and to output a compressed synthesis gas stream comprising said first hydrogen-rich stream and said first feed as a mixed stream or as separate streams,
a methanol synthesis section, arranged to receive at least a portion of said synthesis gas stream, and provide a product stream comprising methanol and an off-gas stream,
a hydrogen recovery section, arranged to receive at least a portion of the off-gas stream from the methanol synthesis section and output a second hydrogen-rich stream and a reject stream, and to recycle at least a portion of the second hydrogen-rich stream to the compression section or to the first hydrogen-rich stream from the electrolysis section.

A process for the production of methanol in the plant described herein is also provided. Said process comprises the steps of:
providing a methanol plant as defined herein;
electrolysing the second feed into a first hydrogen-rich stream,
feeding said first hydrogen-rich stream and said first feed to the compression section and outputting a compressed synthesis gas stream comprising said first hydrogen-rich stream and said first feed as a mixed stream or as separate streams,
feeding at least a portion of said synthesis gas stream to the methanol synthesis section, and outputting a product stream comprising methanol and an off-gas stream,
feeding at least a portion of the off-gas stream from the methanol synthesis section to the hydrogen recovery section, and outputting a second hydrogen-rich stream and a reject stream, and
recycling at least a portion of the second hydrogen-rich stream with the first hydrogen-rich stream from the electrolysis section to the compression section or to the first hydrogen-rich stream from the electrolysis section.

Further details of the technology are provided in the enclosed dependent claims, figures, and examples.

### LEGENDS

The technology is illustrated by means of the following schematic illustrations, in which:

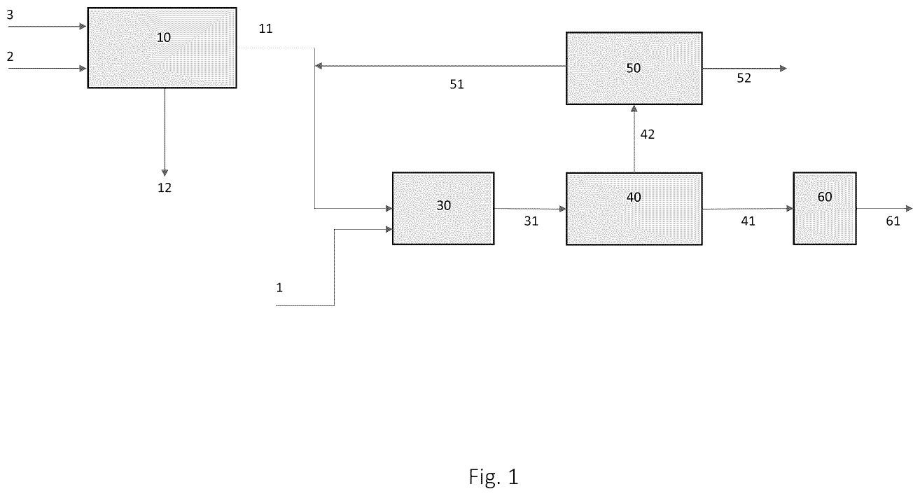

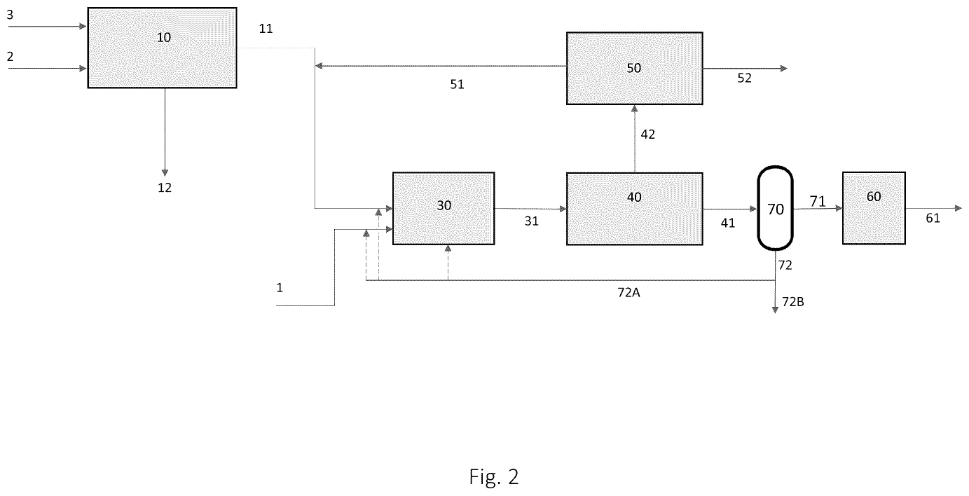

FIGS. 1-2 show layouts for a CO₂-to-MeOH plant according to the invention.

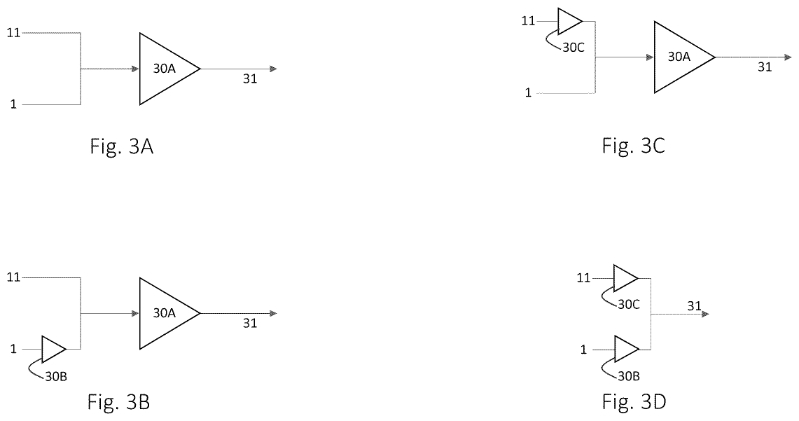

FIG. 3 shows various layouts for a compression section according to the invention.

### DETAILED DISCLOSURE

Unless otherwise specified, any given percentages for gas content are % by volume. All feeds are preheated as required.

The term “synthesis gas” (abbreviated to “syngas”) is meant to denote a gas comprising hydrogen, carbon monoxide, carbon dioxide and small amounts of other gasses, such as argon, nitrogen, methane, etc.

As noted, a methanol plant is provided. A first feed comprising CO₂ is provided to said plant. The first feed suitably comprises more than 90% CO₂, preferably more than 95% CO₂, preferably more than 99% CO₂. The first feed may in addition to CO₂ comprise minor amounts of, for example, steam, oxygen, nitrogen, oxygenates, amines, ammonia, carbon monoxide, and/or hydrocarbons. The first feed suitably comprises only low amounts of hydrocarbon, such as for example less than 5% hydrocarbons or less than 3% hydrocarbons or less than 1% hydrocarbons.

In an embodiment a higher fraction of hydrocarbon can be present in said first feed, such as up to 10% or up to 20%. For high content of hydrocarbons said stream suitably should also contain some steam, which should suitably be added in a ratio of 1-3 relative to the content of hydrocarbons.

In a particular embodiment, the methanol plant further comprises a CO2 electrolysis section arranged to electrolyse a raw stream of CO2 to produce an electrolysed stream comprising CO2 and CO, and wherein said electrolysed stream comprising CO2 and CO is the first feed (1) comprising CO2. By using a combination of electrolysis steps for both a water feed and a CO2 feed, it is possible to form a more reactive synthesis gas for subsequent methanol conversion and/or for production of hydrocarbon products such as synthetic fuels, resulting i.a. in reduction of reactor size such as size of a methanol converter, less formation of water and not least a drastic reduction of the carbon footprint.

Furthermore, savings in terms of hydrogen consumption for particularly methanol conversion are achieved as well. In a particular embodiment, the electrolysed stream comprising CO and CO2, or the synthesis gas, has a molar ratio CO/CO2 in the range 0.2-0.6, such as 0.25 or 0.30 or 0.35, 0.40 or 0.45, 0.50 or 0.55. A synthesis gas having a CO/CO2 in this range, particularly a molar ratio of e.g. 0.55 (i.e. about 65:35 CO2:CO, approximately corresponding to a molar ratio CO2/CO of 1.82), is much more reactive than one based on pure CO2. The cost and the energy consumption of the methanol plant is therefore reduced when using the thus partly converted CO2 stream.

A second feed comprising water is provided to the plant. As this feed is to be electrolysed, it should preferably be high-purity. The second feed suitably comprises more than 99% H₂O preferably more than 99.5% H₂O.

The plant comprises an electrolysis section and a source of electrical power to said electrolysis section. The electrolysis section is arranged to electrolyse the second feed into a first hydrogen-rich stream. The electrolysis section suitably comprises one or more Solid OXIDE Electrolysis Cells (SOECs).

SOECs can be used for direct electrochemical conversion of steam (H₂O), into hydrogen (H₂). The splitting of H₂O occurs at solid oxide electrolysis cell (SOEC) electrodes. Multiple cells are combined into SOEC stacks, and multiple stacks are in turn combined into an SOEC plant. The fuel (H₂O) enters the process side of the SOEC where it is (partly) converted into the product (H₂). The oxygen produced in the conversion on the fuel side is transferred through the electrochemical cell to the oxy side of the SOEC, where it is recombined as gaseous oxygen. It is typically transported away from the SOEC with a flush fluid. Alkaline and PEM electrolysis could be used as an alternative to an SOEC. Such technologies are standard, and need not be described in further detail.

The electrolysis also produces an oxygen stream along with the hydrogen-rich stream. This oxygen stream is typically exported from the plant.

The term “hydrogen rich” meaning that the major portion of the first hydrogen-rich stream is hydrogen, i.e. over 75%, such as over 85%, preferably over 90%, more preferably over 95%, even more preferably over 99% of this stream is hydrogen. In addition to hydrogen, this stream may for example comprise steam, nitrogen, argon, carbon monoxide, carbon dioxide, and/or hydrocarbons. In some cases, a minor content of oxygen may be present in this stream, typically less than 100 ppm. This stream suitably comprises only low amounts of hydrocarbon, such as for example less than 5% hydrocarbons or less than 3% hydrocarbons or less than 1% hydrocarbons.

A compression section is arranged to receive said first hydrogen-rich stream and said first feed and to output a compressed synthesis gas stream comprising said first hydrogen-rich stream and said first feed as a mixed stream or as separate streams. In general, a stream can only be mixed into a stream with a lower pressure, and the mixed gas will have the pressure of the stream with the lowest pressure of the two streams.

In one aspect (illustrated in FIG. 3A) the compression section is arranged to mix the first hydrogen-rich stream and the first feed together to a combined stream and compress the combined stream so as to output a compressed synthesis gas stream. This aspect is most beneficial when the first hydrogen-rich stream and the first feed are at a similar (low) pressure, e.g., below 30 bar.

In another aspect (illustrated in FIG. 3B and FIG. 3C) the compression section is arranged to compress the first hydrogen-rich stream and/or the first feed separately, and then to mix the compressed first hydrogen-rich stream and/or compressed first feed. This aspect is useful when the first hydrogen-rich stream and the first feed are at different pressures. The lower-pressure stream can be compressed to match the pressure of the higher-pressure stream, and then the two streams can be combined. Further additional compression of the mixed, compressed first hydrogen-rich stream and the compressed first feed, may take place, so as to output a compressed synthesis gas stream.

In a further aspect (illustrated in FIG. 3D) the first hydrogen-rich stream and the first feed may be compressed separately before being mixed and sent to the methanol section.

The compressed synthesis gas stream outputted from the compression section suitably has a pressure of above 80 bar, and is thus suitable for use in the methanol synthesis section.

The synthesis gas stream thus provided comprises a high content of CO, CO₂ and H₂ and can further comprise CH₄, N₂, and Ar. Accordingly, the synthesis gas stream provides the components for methanol synthesis. The synthesis gas stream suitably has the following dry gas composition by volume:
70-80% H₂
20-30% CO₂

Small amounts of impurities such as CO, N₂, O₂, Ar, CH₄ etc. may also be present.

### Methanol Synthesis Section

A methanol synthesis section is arranged to receive at least a portion of said synthesis gas stream and provide a product stream comprising methanol and an off-gas stream. The methanol synthesis section thus converts the synthesis gas stream to a raw methanol stream and an off-gas stream. The methanol synthesis reactor in the methanol synthesis section accommodates the following two reactions:

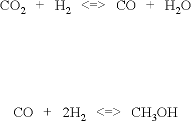

The process of converting the compressed synthesis gas stream can occur, for example by sending the compressed gas through a boiling water reactor, where at least a portion of the synthesis gas is converted to methanol followed by condensation and separation of the methanol in liquid phase, where the methanol in liquid phase therefrom is comprised in the methanol stream. The off-gas stream is produced in this process.

The raw methanol product stream comprises a major portion of methanol; i.e. 60-65% methanol and 35-40% H₂O by weight. Other minor components of this stream include but not limited to, higher alcohols, ketones, aldehydes, dimethyl ether (DME), organic acids and dissolved gases.

To obtain an optimized yield in the methanol production, the stoichiometry of H₂, CO and CO₂ needs to be considered. In a preferred embodiment, the stoichiometry of H₂, CO and CO₂ in the synthesis gas stream falls within an interval such that the synthesis gas stream has a module between 1.8 and 2.2, preferably between 1.9 and 2.1, where the module is defined in terms of molar content:

$$M = ( {H}_{2} - {CO}_{2} ) / ( CO + {CO}_{2} ) .$$

The module of the synthesis gas stream may be adjusted by addition of a (further) hydrogen-rich stream, which is optionally arranged to be admixed with the synthesis gas stream. The hydrogen-rich stream can be provided by an external feed of hydrogen.

From the methanol synthesis section at least a portion of the product stream comprising methanol is suitably fed to a distillation section, said distillation section being arranged to upgrade this product stream to a purified methanol product stream of the required grade, e.g. >95%, >98% or >99% methanol. Therefore, the methanol plant may comprise a distillation section, said distillation section being arranged to receive the product stream comprising methanol from the methanol synthesis section and output a purified methanol product stream.

The off-gas stream from the methanol synthesis section typically comprises: 85-90% H₂, 5-10% CO₂, 0-3% CO.

The methanol plant further comprises a hydrogen recovery section, arranged to receive at least a portion of the off-gas stream from the methanol synthesis section and output a second hydrogen-rich stream and a reject stream, and to recycle at least a portion of the second hydrogen-rich stream to the compression section or to the first hydrogen-rich stream from the electrolysis section.

The hydrogen recovery section can utilise several technologies for separation of gases. Thus, the hydrogen recovery section can comprise a pressure swing adsorption unit, a hydrogen separation membrane or combinations thereof. Independently of the technology applied, the provided second hydrogen-rich stream comprises primarily hydrogen preferably more than 80% more preferably more than 90% hydrogen.

The reject stream from this hydrogen recovery section typically comprises 50-80% H₂, 10-40% CO₂, 0-15% CO, 0-10% CH₄, depending on the configuration.

In a preferred embodiment, the methanol plant comprises a low-pressure separator. A low-pressure separator is a vessel which separates a two-phase flow into a gas stream and a liquid stream. Typically, a low-pressure separator operates at a lower pressure than the high-pressure separator typically found inside a methanol synthesis unit.

The low-pressure separator is arranged to receive at least a portion of the product stream comprising methanol from the methanol synthesis section and to output a purified methanol stream and a carbon-rich stream. Preferably, at least a portion of said carbon-rich stream is arranged to be recycled, preferably wherein at least a first portion of said carbon-rich stream is arranged to be recycled to the compression section or to the first feed or to the first hydrogen-rich stream. The carbon rich stream can in principle be added to both the first feed comprising CO₂ or the hydrogen-rich stream depending on the pressures of these vis-à-vis the carbon-rich stream.

If required, a booster compressor may be arranged to compress the first portion of the carbon-rich stream before it is recycled to the compression section or to the first feed or to the first hydrogen-rich stream.

A process for the production of methanol in the plant described herein is also provided. This process comprises the steps of:
providing a methanol plant as defined herein;
electrolysing the second feed into a first hydrogen-rich stream,
feeding said first hydrogen-rich stream and said first feed to the compression section and outputting a compressed synthesis gas stream comprising said first hydrogen-rich stream and said first feed as a mixed stream or as separate streams,
feeding at least a portion of said synthesis gas stream to the methanol synthesis section, and outputting a product stream comprising methanol and an off-gas stream,
feeding at least a portion of the off-gas stream from the methanol synthesis section to the hydrogen recovery section, and outputting a second hydrogen-rich stream and a reject stream, and
recycling at least a portion of the second hydrogen-rich stream with the first hydrogen-rich stream from the electrolysis section to the compression section or to the first hydrogen-rich stream from the electrolysis section.

All details of the methanol plant described herein are also relevant to the process described herein, mutatis mutandis.

### Specific Embodiments of the Invention

FIG. 1 shows one layout of a methanol plant according to the invention. The methanol plant comprises a first feed (1) comprising CO₂ to said plant and a second feed (2) comprising water to said plant. The methanol plant further comprises an electrolysis section (10) and a source of electrical power (3) to the electrolysis section (10). The electrolysis section (10) is arranged to electrolyse the second feed (2) into a first hydrogen-rich stream (11). A compression section (30) is arranged to receive the first hydrogen-rich stream (11) and the first feed (1) and to output a compressed synthesis gas stream (31) comprising said first hydrogen-rich stream (11) and said first feed (1) as a mixed stream or as separate streams. A methanol synthesis section (40) is arranged to receive at least a portion of the synthesis gas stream (31) and provide a product stream (41) comprising methanol and an off-gas stream (42). A hydrogen recovery section (50) is arranged to receive at least a portion of the off-gas stream (42) from the methanol synthesis section (40) and output a second hydrogen-rich stream (51) and a reject stream (52), and to combine at least a portion of the second hydrogen-rich stream (51) with the first hydrogen-rich stream (11) from the electrolysis section (10).

FIG. 2 shows a layout according to the invention, similar to that in FIG. 1. The methanol plant according to this embodiment additionally comprises a low-pressure separator (70) arranged to receive at least a portion of the product stream (41) comprising methanol from the methanol synthesis section (40) and to output a purified methanol stream (71) and a carbon-rich stream (72). As shown, at least a portion of the carbon-rich stream (72) is arranged to be recycled. At least a first portion (72A) of said carbon-rich stream (72) is arranged to be recycled to the compression section (30) or to the first feed (1) or to the first hydrogen-rich stream (11).

FIGS. 3A-3D show various layouts for a compression section according to the invention.

In FIG. 3A, the compression section is arranged to mix the first hydrogen-rich stream (11) and the first feed (1) together to a combined stream and compress the combined stream in a first compressor (30A) so as to output a compressed synthesis gas stream (31) as a mixed stream.

In FIG. 3B, the compression section is arranged to compress the first feed (1) separately in a second compressor (30B), and then to mix the first hydrogen-rich stream (11) with the compressed first feed (1). Additional compression of the mixed streams takes place in first compressor (30A).

In FIG. 3C, the compression section is arranged to compress the first hydrogen-rich stream (11) separately in a third compressor (30C), and then to mix the first feed (1) with the compressed first hydrogen-rich stream (11). Additional compression of the mixed streams takes place in first compressor (30A).

In a further aspect (illustrated in FIG. 3D) the first hydrogen-rich stream (11) and the first feed (1) may be compressed in separate second (30B) and third (30C) compressors before being mixed and sent to the methanol section.

The standard solution in a methanol plant is to send both off-gas and the carbon-rich stream from the methanol synthesis out of the system. The pressure of the off-gas is high, typically above 40 bar g. The off-gas contains primarily H₂, CO and CO₂ and traces of inerts such as N₂ and CH₄. The pressure of the carbon-rich stream is typically around 4 bar g and contains primarily CO₂, secondarily H₂, with traces of CO, methanol and CH₄.

In a traditional natural gas-based methanol plant, it is not cost-efficient to recycle the carbon-rich stream because the pressure is much lower than the feed gas to the make-up compressor, being around 28 bar g in a traditional natural gas-based methanol plant. However, with the feedstocks being CO2 (1) and water (2) the pressures of the CO₂ (1) and H₂ (11) streams can be as low 0-1 bar g. Therefore, the carbon-rich stream (72) can easily be routed to the suction side of the compression section (30) for further utilization of valuable carbon compounds, thereby reducing emission of carbon compounds to the atmosphere from the methanol plant.

Referring to FIG. 1 (embodiments 1 and 2 in Table 1) at least a portion of the off-gas stream (42) from the methanol synthesis section (40) is directed to a hydrogen recovery section (50), from where a second hydrogen-rich stream (51) can be extracted. The second hydrogen-rich stream (51) is routed to the suction side of the compression section (30). Depending on the type of hydrogen recovery unit, typically either a pressure-swing-adsorption (PSA, embodiment 1) or a membrane type (embodiment 2), the hydrogen rich stream will contain essentially pure H₂ (PSA, embodiment 1) or primarily H₂, secondly CO₂ with traces of CO (membrane, embodiment 2). Oxygen stream (12) generated in the electrolysis section (10) is typically exported from the plant.

Table 1 shows various process parameters of a simulation for processes carried out in the methanol plant of the invention (Embodiments 1-4) as compared to a CO₂- and water-based methanol plant with no recycle of off-gas from a hydrogen recovery section (HRS) and no recycle of a carbon-rich stream from a low-pressure separator (eMEOH BASE). Referring to Table 1 it is seen for embodiment 1 and 2, respectively, that the required hydrogen feed originating from the electrolysis reduces to 98.4% of the standard (eMEOH BASE), while maintaining the overall production of methanol, thereby reducing the power required in the electrolysis to 98.4% of the standard. This takes place with essentially unchanged power consumption for the compression section (30), and unchanged size of the methanol synthesis section (40). Depending on the capacity of the plant this can have a huge impact of the capacity expenditures as well as the operating expenditures of the electrolysis since installation cost of the electrolysis can be high and electrical power expensive. For the methanol plant capacity shown in Table 1, which is considered a medium scale plant based on electrolysis and CO₂, the power savings would be around estimated 5 MW for a typical power consumption of 5 kWh per Nm³ of produced H₂ from the electrolysis.

It is noted that for embodiment 2 the CO₂ feedstock required is also reduced (to 99.6% of standard (eMEOH BASE)) because—as mentioned—the second hydrogen-rich stream (51) also contains CO₂ and CO besides H₂. In principle, this would reduce the size and thereby capacity and operating expenditures of a CO₂ capture unit if the CO₂ feedstock (1) originates from a unit operating on flue gas containing CO₂. If the CO₂ originates from a pipeline network the operating expenditures would also reduce due to the lower CO₂ flow requirement.

Referring to FIG. 2, embodiments 3 and 4 of the invention comprise extensions of the embodiments 1 and 2, resp. further comprising a low-pressure separator (70), where the carbon-rich stream (72) from the low-pressure separator (70) is routed to the suction side of the compression section (30), i.e. (PSA, embodiment 3) or a membrane type (embodiment 4). The effect in both embodiment 3 and 4 is that both the required H₂ flow from electrolysis, and hence the power required for the electrolysis, and required CO₂ flow are reduced due to the low-pressure carbon-rich gas containing both carbon and hydrogen compounds. Again, essentially with the same power requirement for the compression section and maintaining the overall production of methanol with unchanged size of the methanol synthesis section.

Both from a capacity expenditure point of view and an operation expenditure point of view embodiment 4 seems to be the preferable option because a membrane is typically less expensive compared to a PSA and requires less CO₂ feedstock. The feed flow to the hydrogen recovery unit is essentially unchanged between embodiments 1-4.

TABLE 1

| | | | | | eMeOH | eMeOH with |
|---|---|---|---|---|---|---|
| | | | | | with HRS | HRS |
| | | | | eMeOH with | (PSA) + | (membrane) + |
| | | | eMeOH with | HRS | Carbon-rich | Carbon-rich |
| | | | HRS (PSA) | (membrane) | stream | stream |
| | | eMeOH | Embodiment | Embodiment | Embodiment | Embodiment |
| | | BASE | #1 | #2 | #3 | #4 |
| CO2 | Nm3/h | 20915 | 20915 | 20825 | 20625 | 20536 |
| | | (100%) | (100%) | (99.6%) | (98.6%) | (98.2%) |
| H2 from | Nm3/h | 62367 | 61348 | 61340 | 61144 | 61141 |
| electrolyzer | | (100%) | (98.4%) | (98.4%) | (98.0%) | (98.0%) |
| Hydrogen | Nm3/h | 1304 | 1305 | 1306 | 1312 | 1316 |
| recovery feed | | | | | | |
| flow | | | | | | |
| Methanol | Metric | 695.6 | 695.6 | 695.6 | 695.6 | 695.6 |
| production | tonnes | | | | | |
| (as 100% | per day | | | | | |
| methanol) | | | | | | |
| Compression | MW | 14.91 | 14.87 | 14.87 | 14.87 | 14.87 |
| power, total | | (100%) | (99.7%) | (99.7%) | (99.7%) | (99.7%) |
| Estimated | MW | 311.84 | 306.74 | 306.70 | 305.72 | 305.71 |
| electrolyzer | | (100%) | (98.4%) | (98.4%) | (98.0%) | (98.0%) |
| power (at 5 | | | | | | |
| kWh/NM3 H2) | | | | | | |
| Power saving | MW | N/A | 5.10 | 5.14 | 6.12 | 6.13 |
| in electrolyzer | (vs BASE) | | | | | |

The present invention has been described with reference to a number of embodiments and figures. However, the skilled person is able to select and combine various embodiments within the scope of the invention, which is defined by the appended claims.

1. A methanol plant, said methanol plant comprising:
a first feed comprising CO₂ to said plant,
a second feed comprising water to said plant,
an electrolysis section and a source of electrical power to said electrolysis section, said electrolysis section being arranged to electrolyse the second feed into a first hydrogen-rich stream,
a compression section arranged to receive said first hydrogen-rich stream and said first feed and to output a compressed synthesis gas stream comprising said first hydrogen-rich stream and said first feed as a mixed stream or as separate streams,
a methanol synthesis section, arranged to receive at least a portion of said synthesis gas stream, and provide a product stream comprising methanol and an off-gas stream,
a hydrogen recovery section, arranged to receive at least a portion of the off-gas stream from the methanol synthesis section and output a second hydrogen-rich stream and a reject stream, and to recycle at least a portion of the second hydrogen-rich stream to the compression section or to the first hydrogen-rich stream from the electrolysis section.

2. The methanol plant according to claim 1, further comprising a distillation section, said distillation section being arranged to receive the product stream comprising methanol from the methanol synthesis section and output a purified methanol product stream.

3. The methanol plant according to claim 1, wherein the hydrogen recovery section comprises a pressure swing absorption unit, a membrane separation unit, or a combination thereof.

4. The methanol plant according to claim 1, wherein the methanol plant comprises a low-pressure separator arranged to receive at least a portion of the product stream comprising methanol from the methanol synthesis section and to output a purified methanol stream and a carbon-rich stream.

5. The methanol plant according to claim 4, further comprising a booster compressor, being arranged to compress the first portion of said carbon-rich stream before it is recycled to the compression section or to the first feed or to the first hydrogen-rich stream.

6. The methanol plant according to claim 1, wherein the compression section is arranged to mix the first hydrogen-rich stream and the first feed together to a combined stream, and to compress the combined stream so as to output a compressed synthesis gas stream.

7. The methanol plant according to claim 1, wherein the compression section is arranged to compress the first hydrogen-rich stream and/or the first feed separately, and then to mix the compressed first hydrogen-rich stream and/or compressed first feed, optionally with additional compression of the mixed, compressed first hydrogen-rich stream and the compressed first feed, so as to output a compressed synthesis gas stream.

8. The methanol plant according to claim 1, further comprising a CO₂ electrolysis section arranged to electrolyse a raw stream of CO₂ to produce an electrolysed stream comprising CO₂ and CO, and wherein said electrolysed stream comprising CO₂ and CO is the first feed comprising CO₂.

9. A process for the production of methanol in the plant according to claim 1, said process comprising the steps of:
providing the methanol plant according to any one of the preceding claims;
electrolysing the second feed into a first hydrogen-rich stream,
feeding said first hydrogen-rich stream and said first feed to the compression section and outputting a compressed synthesis gas stream comprising said first hydrogen-rich stream and said first feed as a mixed stream or as separate streams,
feeding at least a portion of said synthesis gas stream to the methanol synthesis section, and outputting a product stream comprising methanol and an off-gas stream,
feeding at least a portion of the off-gas stream from the methanol synthesis section to the hydrogen recovery section, and outputting a second hydrogen-rich stream and a reject stream, and
recycling at least a portion of the second hydrogen-rich stream with the first hydrogen-rich stream from the electrolysis section to the compression section or to the first hydrogen-rich stream from the electrolysis section.
